(19) United States
(12) Patent Application Publication
(10) Pub. No.: US 2026/0248867 A1
(43) Pub. Date: Aug. 27, 2026

(54) HETEROTRIMER G-RECEPTOR AGONIST COMPOSITION, AND USES THEREOF

(71) Applicant: MOF Science, LLC, Fort Lauderdale, FL (US)

(72) Inventors: Robert Schaefer, Plano, TX (US); Richard Todd Ullom, Fort Lauderdale, FL (US)

(21) Appl. No.: 19/546,084

(22) Filed: Feb. 20, 2026

Related U.S. Application Data
(60) Provisional application No. 63/761,496, filed on Feb. 21, 2025.

(51) Int. Cl. A61K 36/24; A61K 31/164; A61K 31/4375; A61P 3/04; A61P 3/06; A61P 3/10; A61P 29/00

(52) U.S. Cl. CPC A61K 36/24; A61K 31/164; A61K 31/4375; A61P 3/04; A61P 3/06; A61P 3/10; A61P 29/00

(57) ABSTRACT

Provided herein are compositions, and methods of using the composition as a novel alternative combination therapy, for the preventing, treating, ameliorating, reducing the risks of metabolic syndrome or metabolic diseases or disorders. The composition comprises Oleoylethanolamide, Berberine, and Gymnema sylvestre and targets three different hormone receptors, (1) Glucagon-like peptide 1 (GLP-1) receptors, (2) Glucagon (GCG) receptors, and (3) Glucose-dependent Insulinotropic Polypeptide (GIP) receptors.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/761,496, entitled “THE ROLE OF SYNERGIC ACTION OF OLEOYLETHANOLAMIDE, BERBERINE AND GYMNEMA SYLVESTRE IN GLP-1 RELEASE AND METABOLIC SYNDROME CONTROL”, filed on Feb. 21, 2025. The contents of the above referenced application are incorporated herein by reference in its entirety.

### FIELD OF THE INVENTION

The present invention relates to non-peptide composition acting as an alternative Glucagon-like Peptide 1 (GLP-1)-glucagon (CCG)-Glucose-Dependent Insulinotropic Polypeptide (GIP)-triple agonist, and use thereof; more particularly, to a composition of Oleoylethanolamide, Berberine, and Gymnema sylvestre having activities at GLP-1/CCG/GIP receptors, and use thereof, such as in treating metabolic syndrome or reducing the risk of metabolic diseases or conditions.

### BACKGROUND OF THE INVENTION

Glucagon-like Peptide 1 (GLP-1) medications, such as semaglutide, tirzepatide, liraglutide, saxenda, dulaglutide, relate to a class of drugs used to manage type 2 diabetes and obesity. These drugs, acting as GLP-1 receptor agonists, mimic the natural GLP-1 hormone produced in the gut. They work by stimulating insulin release, suppressing glucagon, slowing gastric emptying, and increasing satiety.

Metabolic syndrome is a set of metabolic disorders that can lead to heart disease, obesity, stroke, and type 2 diabetes (nhlbi.nih.gov. May 2022). These conditions typically begin with a change in insulin metabolism, also called insulin resistance. These disorders are increasingly common in the population, not only in the United States but throughout the Western world. Several factors are interconnected, particularly obesity and a sedentary lifestyle, which can contribute to the risk factors associated with metabolic syndrome. These factors include high cholesterol, insulin resistance, and high blood pressure, which can lead to cardiovascular disease and type 2 diabetes. Metabolic syndrome can be a precursor to metabolic diseases, which are the outcome of prolonged metabolic dysfunction.

Because metabolic syndrome and insulin resistance are closely linked, many health professionals believe that insulin resistance may be a cause of metabolic syndrome (Metabolic Syndrome and Insulin Resistance: Underlying Causes and Modification by Exercise Training. Compr Physiol. 2013 January; 3(1):1-58). But to date, there is no direct link between the two conditions. Others believe that hormonal changes caused by chronic stress, particularly changes in cortisol, lead to abdominal obesity, insulin resistance, and higher blood lipids (triglycerides and cholesterol) (Stress and Obesity: Are There More Susceptible Individuals?Curr Obes Rep. 2018 Apr. 16; 7(2):193-203).

Genetic changes, mitochondrial dysfunction, and aging itself are other factors that may contribute to metabolic syndrome (Mitochondrial and metabolic dysfunction in ageing and age-related diseases. Nat Rev Endocrinol. 2022 Feb. 10; 18(4):243-258). In these cases, due to the difficulty in oxidizing fatty acids, the liver may be compromised, triggering hepatic steatosis, and generating more inflammation and insulin resistance. This process becomes a snowball effect, where the excessive formation of triglycerides ends up activating inflammatory triggers, increasing the expression of pro-inflammatory cytokines that can lead to organ failure (Hepatic steatosis, low-grade chronic inflammation and hormone/growth factor/adipokine imbalance. World J Gastroenterol. 2010 Oct. 14; 16(38):4773-4783).

The action of glucagon-like peptide-1 receptor agonists (GLP-1RA) has been attracting the attention of the scientific community in recent years. When released, this peptide has proven effective in controlling metabolic syndrome, especially in controlling blood glucose levels. Some studies have shown that not only drugs can interact with GLP-1RA, but some nutraceuticals have also demonstrated a remarkable ability to control metabolic syndrome with milder side effects. This brings alternative possibilities for controlling metabolism other than through traditional medicine (An overview of glucagon-like peptide-1 receptor agonists for the treatment of metabolic syndrome: A drug repositioning. Iran J Basic Med Sci. 2020 May; 23(5):556-568).

In the same way, gastric inhibitory polypeptide receptor (GIP-R) and glucagon receptor (GCGR) when activated by their respective agonists, also show remarkable results in controlling metabolism. Studies have shown that the synergistic agonist action on the three receptors presents promising results in the treatment of metabolic syndrome (GLP-1, GIP and glucagon receptor poly-agonists: A new era in obesity pharmacotherapy (Obesity (Silver Spring). 2022 Jul. 24; 30(9):1718-1721.).

The human body's cells, especially in the membranes, are equipped with receptors that work like a “lock” so that certain substances can bind and enter their interior, acting on metabolic processes in general. Therefore, each lock has a specific “key” (agonist or ligand) to open certain doors and uniquely act on metabolism. This process has been referred to as the “key-lock” mechanism by some scientists. By knowing the correct key (agonist) that fits the correct lock (receptor), metabolic reactions can be modulated for a specific purpose. This is how the pharmaceutical industry discovered how to use some chemical compounds that had an affinity with certain receptors and could interact inside cells, modulating metabolism in order to control metabolic problems.

One of the best-known processes today is that of semaglutide, which binds to GLP-1 (glucagon-like-peptide-1) receptors, acting both in appetite control and in insulin modulation, improving glucose metabolism and, consequently, weight loss. However, cells have other receptors that can interact differently with similar results, as in the case of tirzepatide (Mounjaro). This substance is a ligand of the GIP (glucose-dependent insulinotropic polypeptide) receptor. Both act as a key in a lock to activate a metabolic cascade in response to the ligand itself. Due to the complexity of nature, receptors can be formed by a single receptor (monomer), two receptors of the same nature (homodimers), two receptors of different natures (heterodimers), or even three receptors of different natures (heterotrimers). Accordingly, double peptide agonists (GLP-1/GIP), such as tirzepatide, and even triple peptide agonists (GLP-1/GIP/CCG), such as retatrutide, are known as well.

While these drugs (GLP agonists and the double agonists, the triple agonists) are effective in managing type 2 diabetes and result in weight loss for those using the drugs to control obesity, there are some potential drawbacks in using them. GLP-1 (as well as double agonists and the triple agonists) the medications can cause various side effects and have been linked to potential long term risks related to gastrointestinal issues (gastroparesis, ileus, intestinal obstructions, gallbladder injures), muscle loss, kidney health, as well and health concerns related to pancreatitis and thyroid tumors. New and more effective GLP-1-like compositions, and method of use, are needed.

### SUMMARY OF THE INVENTION

Embodiments of the invention are directed towards a composition, and therapeutic/medical use of the composition, for controlling metabolic syndrome and/or preventing, treating, ameliorating, or reducing the risk of metabolic diseases or disorders, including diabetes and obesity, as well as for reduction of excess food intake. The composition, and use thereof, comprises Oleoylethanolamide, Berberine, and Gymnema sylvestre, which via synergistic action, act via interacting as a ligand of triple G receptors (Glucagon-like peptide 1 receptor (GLP-IR), Glucagon receptor (GCGR) and Gastric Inhibitory Polypeptide receptor (GIPR)) in the intestinal epithelium.

Accordingly, it is an objective of the invention to teach a composition which acts as triple-agonists.

It is an objective of the invention to teach a composition which acts as triple-agonists activating GLP-1 receptors, GCG receptors, and GIP receptors.

It is a further objective of the invention to provide a composition which functions as a GLP-1/glucagon/GIP receptor agonists for use in the treatment of disorders of the metabolic syndrome.

It is a further objective of the invention to provide a composition which induces sustainable weight loss.

It is a further objective of the invention to provide a composition which improves glycemic control.

It is a further objective of the invention to provide a composition which aids in reducing high cholesterol, high LDL-cholesterol, and triglycerides.

It is a further objective of the invention to provide a composition which may be used to treat or minimize the disease states associated with type 2 diabetes mellitus, metabolic syndrome, non-alcoholic based fatty liver, and obesity.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 illustrates a “key-lock” mechanism of a triple-agonist composition in accordance with embodiments of the invention;

FIG. 2 is a schematic illustration of the binding and possible modes of action associated with receptor binding and potential causes of action;

FIG. 3A is a table providing physiological data of several subjects, pre-administration with a triple-agonist composition in accordance with embodiments of the invention;

FIG. 3B is a table providing metabolic data of the subjects shown in FIG. 3A;

FIG. 4A is a table providing physiological data of the subjects in FIG. 3A and FIG. 3B, post-administration of the triple-agonist composition;

FIG. 4B is a table providing metabolic data of the subjects shown in FIG. 4A;

FIG. 5 is a comparison profile of Patients A, B, C, and D, illustrating differences pre and post-administration of triple-agonist composition in accordance with embodiments of the invention;

FIG. 6 is a comparison profile of Patients A, B, C, and D, illustrating blood panel differences pre- and post-administration of triple-agonist composition in accordance with embodiments of the invention.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

The present invention describes a novel alternative combination therapy, in the form of a unique composition, for the preventing, treating, ameliorating, reducing the risks of metabolic syndrome or metabolic diseases or disorders. As an illustrative example, the present invention has been found to be useful in preventing, treating, ameliorating, or reducing the risk of obesity, type-2 diabetes, or appetite suppression. The triple-agonist composition targets three different hormone receptors, (1) Glucagon-like peptide 1 (GLP-1) receptors, (2) Glucagon (GCG) receptors, and (3) Glucose-dependent Insulinotropic Polypeptide (GIP) receptors. The composition may be used to control metabolic syndrome conditions, or more importantly, in preventing, treating, ameliorating, or reducing the risk of metabolic diseases or disorders via administration or delivery of the composition in effective amounts to a subject in need thereof.

Acting as GLP1-Glucagon-GIP triple agonists, embodiments of the compound may have a combination effect on fat metabolism (glucagon), improved glycemic control (GIP) and on blood glucose levels and food intake (GLP1). As such, embodiments of the composition may therefore act to accelerate elimination of excessive adipose tissue, induce sustainable weight loss, and improve glycemic control. Embodiments of the composition may also act to reduce high cholesterol, high LDL-cholesterol, and triglycerides. Additionally, embodiments of the composition may further be used to treat or minimize the disease states associated with type 2 diabetes mellitus, metabolic syndrome, non-alcoholic based fatty liver, and obesity.

Accordingly, embodiments of the composition may be used (alone or in combination) for preventing weight gain, promoting weight loss, reducing excess body weight or treating obesity (e.g., by control of appetite, feeding, food intake, calorie intake, and/or energy expenditure and lipolysis), including morbid obesity, as well as associated diseases and health conditions including but not limited to obesity linked inflammation, obesity linked gallbladder disease and obesity induced sleep apnea. Embodiments of the composition may also be used for treatment of insulin resistance, glucose intolerance, pre-diabetes, increased fasting glucose, type 2 diabetes, hypertension, dyslipidemia (or a combination of these metabolic risk factors).

Embodiments of the composition may be used (alone or in combination) for the treatment and/or prevention of any of the diseases, disorders, or conditions described herein, including insulin resistance, glucose intolerance, increased fasting glucose, pre-diabetes, type 1 diabetes, type 2 diabetes, gestational diabetes hypertension, dyslipidemia, or a combination thereof. In certain embodiments, the blood fat disorder is selected from high triglycerides, low HDL cholesterol, high LDL cholesterol, plaque buildup in artery walls, or a combination thereof. In certain embodiments, the prothrombotic state is selected from high fibrinogen levels in the blood and high plasminogen activator inhibitor-1 levels in the blood. In certain embodiments, the proinflammatory state is an elevated C-reactive protein level in the blood. In certain embodiments, the obesity related disorder is selected from obesity linked inflammation, obesity linked gallbladder disease and obesity induced sleep apnea.

Embodiments of the composition may be used (alone or in combination) for those already using GLP-1 medications or products (including double agonist medications or products or triple agonist medications or products). In this use, embodiments of the composition may be provided as a maintenance dosage(s) provided such subjects in order to wean or take them off of GLP-1 medications or products (or double agonist medications or products or triple agonist medications or products) and/or to reduce any rebound effect associated with subjects stopping those GLP-1 medications or products (or double agonist medications or products or triple agonist medications or products).

Embodiments of the composition may be formulated as pharmaceutical compositions prepared for storage or administration. The compositions may comprise a therapeutically effective amount of the compound as described herein, one or more pharmaceutically acceptable excipients, and/or include one or more of a pharmaceutically acceptable carrier.

In certain embodiments, the pharmaceutical composition may be formulated as a solid composition, such as a pill, a tablet, a capsule, and administered, for example, via oral routes.

In certain embodiments, the pharmaceutical composition is formulated as a liquid. The liquid embodiments may be suitable for administration by injection.

As used herein, the use of a numerical range expressly includes all possible subranges, all individual numerical values within that range, including integers within such ranges and fractions of the values unless the context clearly indicates otherwise. For example, a range of 5-6 includes 5.0, 5.1, 5.2, 5.3, 5.4, 5.5, 5.6, 5.7, 5.8 5.9, and 6.0, or any other fractional value or number within the range. A range of 80-100% would mean, for example, 85%, 90%, 95%, as well as 81%, 82%, 83%, 84%, 85%, 86%, 87%, 88%, 89%, 90%, 91%, 92%, 93%, 94%, 95%, 96%, 97%, 98%, 99%, or 80.5%, 81.5%, 82.5%, 83.5%, 84.5%, 85.5%, 86.5%, 87.5%, 88.5%, 89.5%, 90.5%, 91.5%, 92.5%, 93.5%, 94.5%, 95.5%, 96.5%, 97.5%, 98.5%, or 99.5% (or any other fractional value or number).

As used herein, the term “about” in reference to any numerical values, such as a concentration or a concentration range described herein, is to be understood as a numerical value of ±10% of the recited value. For example, a concentration of 1 mg/mL includes 0.9 mg/mL to 1.1 mg/mL, a concentration of 100 mg/mL includes 90 mg/mL to 110 mg/mL, or a value of 1000 mg includes 900 mg to 1100 mg. A concentration range of 1% to 10% (w/v) includes 0.9% (w/v) to 11% (w/v) A concentration range of 10% to 100% (w/v) includes 9% (w/v) to 110% (w/v).

As used herein, use of the term “subject” means any animal, preferably a mammal, most preferably a human.

As used herein, the use of the term “administering” with respect to the methods of the invention, means a method for therapeutically or prophylactically preventing, treating, ameliorating, or reducing the risk of a syndrome, disorder or disease as described herein by using a composition in accordance with the invention or a form, composition or medicament thereof. Such methods include administering an effective amount of the composition, compound, a form, composition or medicament thereof at different times during the course of a therapy or concurrently in a combination form. The methods of the invention are to be understood as embracing all known therapeutic treatment regimens.

As used herein, the term “delivering” is defined as providing and/or administering the composition described herein by drug administration routes known to one of skill in the art, such as, oral administration of tablets, capsules, liquids, powders, granules, microparticles, sachets, suppositories, etc. which contain the described ingredient(s) and (optionally) one or more carriers and/or diluents and/or adjuvants or other excipients.

As used herein, the term “effective amount” means that amount of composition (which may include the individual components making up the composition) or pharmaceutical agent that elicits the biological or medicinal response in a tissue system, animal or human, which includes preventing, treating, ameliorating, or reducing the risk of a syndrome, disorder, or disease being treated, or the symptoms of a syndrome, disorder or disease being treated.

As used herein, the term a “pharmaceutically acceptable excipient” refers to any ingredient in the pharmaceutical composition which is not the active pharmaceutical ingredient.

For example, the excipient may enhance absorption of the active substance. The excipient may be functional or inert and may serve one or more purposes. The excipient may be a natural or synthetic substance included in the formulation of a composition that is suitable for use in humans and/or non-human mammals without undue adverse side effects, including but not limited to toxicity, irritation or allergic response. Illustrative examples of excipients may include, but are not limited, an isotonicity agent(s), a lubricant(s), a preservative(s) (including anti-oxidant(s), including antimicrobial preservative(s), a carrier(s), a vehicle(s), a filler(s), an anti-adherent(s), an antioxidant(s), a binder(s), a buffer(s), a coating(s), a compression aid(s), a disintegrant(s), a dye(s) (colors), an emollient(s), an emulsifier(s), a filler(s) (diluent(s)), a film former(s) or coating(s), a flavor(s), a fragrance(s), a glidant(s) (flow enhancer(s), a lubricant(s), a flow control agent(s), a crystallization inhibitor(s), a solubilizer(s), a stabilizer(s), a coloring agent(s), a flavoring agent(s), a surfactant(s), printing ink(s), a sorbent(s), a suspending or dispersing agent(s), a solvent(s), a colloid stabilizer(s), a sweetener(s), and water. Excipients may include, but are not limited to: butylated hydroxytoluene (BHT), calcium carbonate, calcium phosphate (dibasic), calcium stearate, carbomer, croscarmellose, crosslinked polyvinyl pyrrolidone, citric acid, crospovidone, cellulose derivatives including ethylcellulose, hydroxypropyl cellulose, hydroxypropyl methylcellulose or hypromellose, docusate sodium, gelatin, gelucire 43/01, lactose, magnesium stearate, maltitol, mannitol, methylcellulose, methyl paraben, microcrystalline cellulose, polyethylene glycol, poly(ethylene oxide), polyvinyl pyrrolidone, povidone, pregelatinized starch, propyl paraben, shellac, silicon dioxide, sodium carboxymethyl cellulose, sodium starch glycolate, sorbitol, starch (corn), stearic acid, sucrose, talc, titanium dioxide, vegetable oils, wax, including white, yellow or bees wax, and xylitol. Excipients may also include diluents (e.g., saline and aqueous buffer solutions), aqueous carriers, and nonaqueous carriers, for example, water, ethanol, polyols (such as glycerol, propylene glycol, polyethylene glycol, and the like), and suitable mixtures thereof, vegetable oils, such as olive oil, and injectable organic esters, such as ethyl oleate. The amount of each excipient used may vary within ranges conventional in the art.

As used herein, the term a “pharmaceutically acceptable carrier or vehicle” refers to any ingredient that function in the delivery of the compound, ensuring safe, stable, and effective delivery of the active ingredients, and may include, but not limited to, Self-Emulsifying Phospholipid Pre-concentrates (SEPPs) (Formulations that spontaneously form emulsions, boosting the absorption of lipophilic nutrients or drugs; (Phospholipid complex such as Liposomes: Spherical vesicles with phospholipid bilayers used to encapsulate drugs); Phytosomes (Complexes of phospholipids with botanical extracts that enhance the bioavailability of polyphenols); Polyether compound such as Polyethylene glycol (PEG), polyethylene oxide (PEO), polyoxyethylene (POE); Cyclic oligosaccharides, such as Cyclodextrins (CDs); Metal-organic-framework, such as magnesium-gallate; Spherical microparticles, such as microspheres; Biomolecules attached to its surface, such as nanoparticle-biomolecule conjugate. These technologies above can be used in all three ingredients in the triple-agonist composition.

As used herein, the term “pharmaceutically acceptable salt” refers to salts that retain the biological effectiveness and properties of a compound, which are not biologically or otherwise undesirable for use in a pharmaceutical. Salts include pharmaceutically acceptable salts, such as, e.g., acid addition salts and basic salts. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, the term “treating” is defined as subjecting a patient to a management regimen for the purpose of treating a disease or disorder and obtaining beneficial or desired results, such as amelioration of disease signs or symptoms or improvement in biochemical, radiological, behavioral or physical markers of disease activity or disease status. Examples of beneficial or desired results can include, but are not limited to resolution of inflammation, resolution of biochemical imbalances, improvement in quality of life, improvement in cognitive and behavioral status, improvement in motor function, improvement in emotional and mood status, sleep improvement, or more generally alleviation or amelioration of one or more symptoms or conditions; diminishment of extent of disease; stabilization of a state of disease; prevention of spread of disease; delay or slowing the progress of the disease; amelioration or palliation of a disease, disorder, or condition; and partial or complete remission of a significant disease manifestation. Treatment may be prophylactic (preventative), palliative, symptomatic, and/or curative.

As used herein, the term “treatment” refers to, one or more of, relieving, alleviating, delaying, reducing, improving, or managing one or more symptoms of a condition in the subject. The term “treatment” may also refer to, one or more of arresting, delaying the onset or reducing the risk of developing or worsening a condition. In certain uses, “treatment” may mean inhibiting or reducing an increase in pathology or symptoms (e.g. weight gain, hyperglycemia) when compared to the absence of treatment, and is not necessarily meant to imply complete cessation of the relevant condition.

As used herein, the term “metabolic syndrome” refers to a group of conditions, occurring together, that increase the likelihood of developing metabolic disease. The conditions, or risk factors include Abdominal adiposity (excess fat around the waist, measured by waist circumference), insulin resistance, Hypertension (HTN), Dyslipidemia (Dyslipidemia includes abnormal cholesterol and lipid levels, such as Hypertriglyceridemia (High levels of triglycerides in the blood), Hypercholesterolemia (Elevated total cholesterol levels) and Low HDL (Low levels of high-density lipoprotein)).

As used herein, the term “metabolic disease or disorder” refers to chronic conditions that result from prolonged metabolic dysfunction, such as Type 2 diabetes (T2DM), Obesity, Atherosclerosis, or Cardiometabolic disease includes conditions such as heart attacks, strokes, and non-alcoholic fatty liver disease (NAFLD), all of which arise from metabolic dysfunction.

The present invention relates to a tri-therapy/tri-component composition acting as GLP-1/Glucagon/GIP receptor agonists and use thereof, for example in the treatment of metabolic disease or disorders and/or related to metabolic syndrome, including diabetes and obesity, as well as for reduction of excess food intake and weight loss. Human cells, especially in the membranes, are equipped with receptors that work like a “lock” so that certain substances can bind and enter their interior, acting on metabolic processes in general. Therefore, each lock has a specific “key” (agonist or ligand) to open certain doors and uniquely act on metabolism.

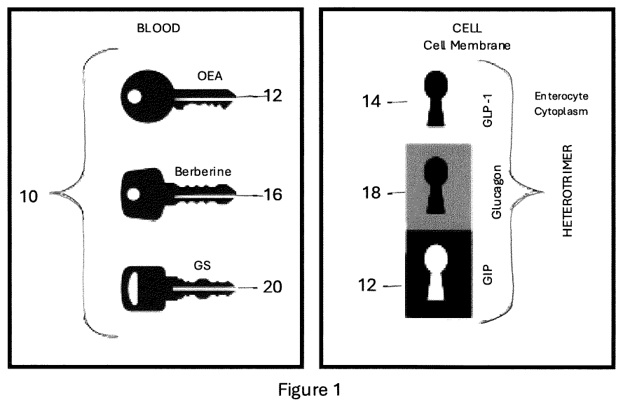

Referring to FIG. 1, as applied to the present invention, this “key-lock” mechanism works by providing the correct key (agonist) fitting the correct lock (receptor), so that metabolic reactions can be modulated for a specific purpose. In this model, the current composition, referred to generally as a triple-agonist composition 10, comprises 1) an effective mount of a first compound, OEA, key 12, to target Glucagon-like peptide 1 (GLP-1) receptors, lock 14, within the cytoplasm of a cell, 2) an effective amount of a second compound, Berberine, its pharmaceutically acceptable salts, or Berberine derivatives, key 16, to target GCG receptors, lock 18, within the cytoplasm of a cell, and 3) an effective mount of a third compound, GS, key 20 that targets Glucose-dependent GIP receptors, lock 22, within the cytoplasm of a cell. The interaction, or binding, of keys 12,16, and 20, to their respective locks 14, 18, and 22 activates one or more metabolic cascade(s).

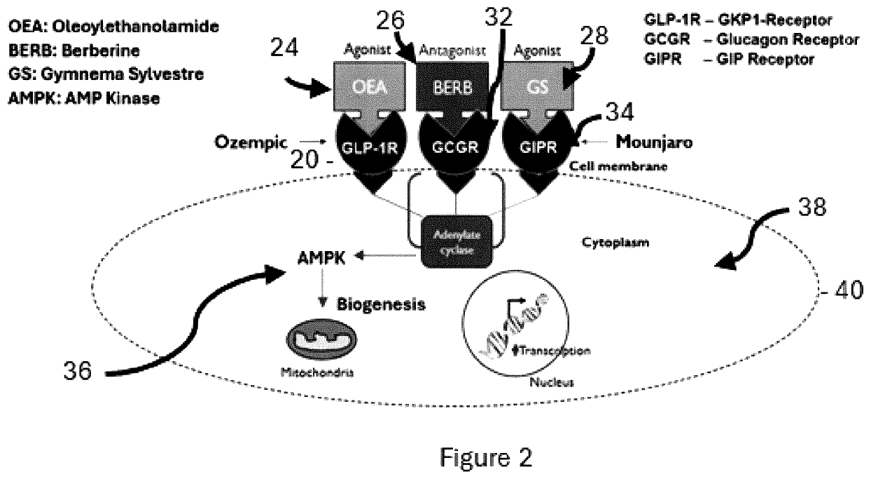

Referring to FIG. 2, the individual components of the triple-agonist composition 10, agonists OEA 24, antagonist Berberine, its pharmaceutically acceptable salts, or Berberine derivatives, 26, and agonist GS 28, are shown bound to cell membrane receptors, GLP-1R 30, GCG-R 32, and GIPR 34, respectively. The binding of these ligands activates metabolic pathways/cascades 36 (such as stimulate metabolic pathways involved in controlling aging, such as the phosphorylation of the AMP-activated protein kinase (AMPK), increase mitochondrial biogenesis by simultaneously increasing the activity of PPAR-alpha and AMPK, or increase fatty acid oxidation by increasing the activity of the carnitine palmitoyltransferase 1 and 2 complex (CPT1 and CPT2), which uses carnitine to transport fatty acids into the mitochondrial matrix, activating beta-oxidation within the cytoplasm 38 of the cell 40) to provide a physiological effect or cell action.

Oleoylethanolamide (OEA), natural or synthetically derived: OEA, IUPAC name (9Z)-N-(2-Hydroxyethyl)octadec-9-enamide, is an endogenous peroxisome proliferator-activated receptor alpha (PPAR-α) agonist.

Since OEA has a high-affinity endogenous ligand for PPAR-α, it may regulate glucose homeostasis by increasing insulin sensitivity in adipose and muscle tissue, control the expression of genes involved in S-cell function, and enhance fatty acid oxidation and reduce fat storage. This role of OEA in metabolism is believed to play a role in helping with non-alcoholic fatty liver disease and liver inflammation. OEA may bind not only to G-protein coupled receptor 119 (GPR119) receptors on intestinal L-cells but also in pancreatic β-cells, stimulating the secretion of glucagon-like peptide-1 (GLP-1). OEA may increase insulin sensitivity and glucose uptake into tissues, promoting direct glucose excretion, reducing insulin resistance through physiological (not pharmaceutical) mechanisms. Another, no less important action in metabolism is its interaction with brain metabolism. OEA may be useful in signaling sufficient food has been consumed by engaging satiety receptors PPAR-α, Transient Receptor Potential Vanilloid 1 (TRPV1), and GPR119. It may also modulate dopamine signaling in brain regions linked to emotional eating and cravings, reducing reward-driven overeating, and increasing resting energy expenditure and fat oxidation. All these interactions with specific receptors mentioned above, combined with the metabolic control of markers such as tumor necrosis factor alpha (TNF-α), interleukin-6 (IL-6), and C-reactive protein (CRP), make OEA an essential ingredient in the triple-agonist composition 10 for appetite control, reduction of fatty liver, reduction of dyslipidemia, reduction of body fat, and consequently, weight loss.

In certain embodiments, the amount/concentration of OEA in the triple-agonist composition 10 may be from about 100 mg to about 600 mg.

In certain embodiments, the dosage of OEA, its pharmaceutically acceptable salts, may be administered once a day.

In certain embodiments, the dosage of OEA, its pharmaceutically acceptable salts, may be administered twice a day.

In certain embodiments, the dosage of OEA, its pharmaceutically acceptable salts, may be administered three times a day.

Berberine, its pharmaceutically acceptable salts, or Berberine derivatives: Berberine is a quaternary ammonium salt from the protoberberine group of isoquinoline alkaloids. It has a IUPAC name of (5,6-Dihydro-9,10-dimethoxybenzo[g]-1,3-benzodioxolo[5,6-a]quinolizinium), with a chemical formula of:

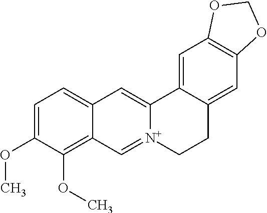 [SMILES: COc1ccc2cc3[n+](cc2c1OC)CCc1cc2c(cc1-3)OCO2]

Berberine is found in various plant species of Berberis (e.g. Berberis aquifolium (Oregon grape), Berberis vulgaris (Barberry), and Berberis aristata (Tree Turmeric)), as well as other plant families, including but not limited to Hydrastis canadensis (Goldenseal), Phellodendron amurense (Amur Cork Tree, Huang Bai, Huang Po, Po Mu) and Coptis chinensis (Chinese Goldthread, Huang-Lian, Huang-Lien), and Tinospora cordifolia, and to a smaller extent in Argemone mexicana (Prickly Poppy) and Eschscholzia californica (Californian Poppy), Rhizoma coptidis Huanglian Jiedu decoction, San-Huang-Xie-Xin-Tang, Xietianwu, Gegen Quinlian, and Shizhu. Berberine is primarily isolated from the roots, rhizomes, stems, and bark.

In accordance with the present invention, berberine, berberine derivatives, or its salts may include, but are not limited to, berberine alkaloid, berberine base, berberinehydrochloride, dihydroberberine (DHB), berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine, tetrahydroprotoberberine 8-cyanodihydroberberine, dimeric protoberberine alkaloids, demethylated protoberberine alkaloids, quataternary protoberberine alkaloids, protoberberineand protoberberine alkaloids, the salts of berberine, including berberine hydrochloride, berberine chloride, berberine sulfate, berberine tannate and other salts known to one of skill in the art.

Berberine, berberine derivatives, or its salts activate adenosine monophosphate-activated protein kinase (AMPK), a master regulator of cellular energy metabolism. This activation promotes glucose and fatty acid utilization, inhibits de novo lipogenesis (fat synthesis), and normalizes lipid, glucose, and energy imbalances. Both can improve glucose metabolism, such as enhancing insulin sensitivity and reducing insulin resistance (HOMA-IR). It promotes insulin secretion from pancreatic β-cells through cAMP signaling pathways, increases glucose uptake via GLUT4 translocation to cell membranes, and inhibits gluconeogenesis (glucose production) in the liver. These processes work together to reduce triglycerides (TG) and total cholesterol (TC), decrease LDL cholesterol, increase HDL cholesterol, stabilize LDL receptor (LDLR) mRNA, and increase plasma LDL clearance through receptor-mediated endocytosis. Studies have also shown that berberine reduces apolipoprotein B and hs-CRP compared to a placebo. Berberine, berberine derivatives, or its salts may act as modulators of glycemia, improving glucose metabolism in general.

In certain embodiments, the amount/concentration of Berberine, its pharmaceutically acceptable salts, or Berberine derivatives, in the triple-agonist composition 10 may be from about 50 mg to about 1200 mg.

In certain embodiments, the dosage of Berberine, its pharmaceutically acceptable salts, or Berberine derivatives may be administered once a day.

In certain embodiments, the dosage of Berberine, its pharmaceutically acceptable salts, or Berberine derivatives may be administered twice a day.

In certain embodiments, the dosage of Berberine, its pharmaceutically acceptable salts, or Berberine derivatives may be administered three times a day.

Gymnema sylvestre: Gymnema sylvestre is a perennial woody vine native to Asia (including the Arabian Peninsula), Africa and Australia. It has been used in Ayurvedic medicine. Common names include Gymnema, Australian cowplant, and Periploca of the woods, and the Hindi term gurmar, which means “sugar destroyer”. The leaves and extracts contain gymnemic acids, the major bioactive constituents that interact with taste receptors on the tongue to temporarily suppress the taste of sweetness.

Gymnema sylvestre may help in stabilizing blood glucose through several pathways, among them reducing intestinal glucose absorption through Gymnemic acids, which compete with glucose for intestinal receptors, enhancing insulin secretion by stimulating insulin release from pancreatic β-cells, increasing insulin sensitivity, which improves cellular glucose uptake, and inhibiting liver glucose release by blocking adrenaline-stimulated hepatic glucose output. GS may play a role in decreasing body weight, reduced BMI, reduced triglycerides, LDL and VLDL cholesterol, and increased HDL cholesterol. GS is known to have antioxidant activity by reducing tissue malondialdehyde levels and maintaining glutathione and catalase. GS may be a metabolic modulator in both glycemic control and obesity management.

GS Forms: In certain embodiments, GS may be in a raw powder form, such as from dried leaf, from dried stem, or combinations thereof. In certain embodiments, GS may be a liquid extract.

In accordance with certain embodiments, GS, regardless in powder form or liquid form, is standardized for Gymnemic acid to ensure consistent concentrations of Gymnemic acid. In certain embodiments, GS is standardized between about 1% to about 99% Gymnemic acid, expressed as mg of extract per capsule, or mg of Gymnemic acid per serving/dose. In certain embodiments, GS is standardized between about 25% to about 75%, expressed as mg of extract per capsule, or mg of Gymnemic acid per serving/dose. Accordingly, in certain embodiments, GS is standardized to about 25% Gymnemic acid per serving/dose. In certain embodiments, GS is standardized to about 50% Gymnemic acid per serving/dose. In certain embodiments, GS is standardized to about 75% Gymnemic acid per serving/dose. In certain embodiments, GS is standardized to greater than 75% Gymnemic acid per serving/dose.

In certain embodiments, the triple-agonist composition 10 may conspire effective amounts of Gymnemic acid (CAS No: 122168-40-50), a triterpone saponin (such as isolated, in purified form, using known extraction and purification methods). In certain embodiments, Gymnemic acid may have a purity of about 25%-100%, such as greater than or equal to 95%, 80-90%, or 25-75%. Accordingly, in certain embodiments, the triple-agonist composition 10 may include OEA, Berberine, (its pharmaceutically acceptable salts, or Berberine derivatives) and Gymnemic acid.

In certain embodiments, the amount/concentration of GS, in the triple-agonist composition 10 may be from about 200 mg to about 900 mg.

In certain embodiments, the dosage of GS, or GS derivatives may be administered once a day.

In certain embodiments, the dosage of GS may be administered twice a day.

In certain embodiments, the dosage of GS may be administered three times a day.

Combination Action Of The Triple-Agonist Composition: The role of OEA in appetite control through PPAR-α, TRPV1, and GPR119 receptors, as well as in the activation of CPT 1 and 2 in fatty acid oxidation, brings metabolism to a plateau in the control of metabolic syndrome. However, other metabolic pathways must be adjusted for the therapeutic potential to be successfully achieved. The desensitization of insulin receptors exerted by GS, as well as its inhibition of glucose entry into the bloodstream through enterocytes, is believed to enhance the role of OEA in human metabolism.

Glycemic control by GS, along with increased fatty acid oxidation by OEA, may allow for a decrease in systemic triglyceride levels, which could lead to a reduction in inflammatory processes and, consequently, a recovery of homeostasis in general. However, signaling of certain proteins would still be necessary.

The presence of berberine in the triple-agonist composition 10 may play a promising role in activating both AMPK and GLUT4, an essential protein mainly found in muscle and adipose tissue, responsible for transporting glucose from the blood into cells. Therefore, not only does glucose oxidation become more efficient, but also fatty acid oxidation. The phosphorylation of AMPK by berberine may allow for an increase in mitochondrial biogenesis, working synergistically with OEA so that more healthy mitochondria are able to oxidize fatty acids. Once oxidative processes are controlled and mitochondrial function is restored, metabolic gain becomes exponentially more productive, minimizing the risks of metabolic syndrome. Such benefits could not be achieved by the ingredients/compounds in isolation, but their synergistic action in combination.

The components of the triple-agonist composition 10 are believed to act in the following: 1) OEA, which, in addition to being an appetite modulator, increases the mitochondria's ability to oxidize fatty acids, decreasing triglyceride levels; 2) Berberine, also assisting in the desensitization of insulin receptors, but mainly in the phosphorylation of AMPK, demonstrating metabolic control through increased mitochondrial biogenesis; and 3) GS, controlling glucose absorption by the small intestine and desensitizing insulin receptors in the cell membrane, which would result in less insulin synthesis and, therefore, less triglyceride synthesis;

Thus, the presence of these three triple G receptor agonists is believed to promote a metabolic action not in an arithmetic progression but rather in a geometric progression in the body's biochemistry, making it a promising compound for future therapies in the control of metabolic syndrome.

Synergy of the OEA, Berberine, and GS: Metabolic syndrome is not a single-pathway problem. It is a network disorder involving insulin resistance, impaired GLP-1 signaling, hepatic fat accumulation, dyslipidemia, chronic inflammation, appetite dysregulation, and reward-driven overeating. Because the dysfunction is multi-layered, single-pathway solutions often produce incomplete results.

The triple-agonist composition 10, namely the combination of OES, Berberine, and Gymnema sylvestre works at complementary control points across metabolism. When combined, they create metabolic reinforcement loops that amplify outcomes beyond what each ingredient could achieve alone.

### GLP-1 Pathway+Appetite Regulation:

### OEA: Endogenous GLP-1 Support+Satiety Signaling

OEA may stimulate GLP-1 release through GPR119 activation while simultaneously activating PPAR-ax and TRPV1. This action may result in increased endogenous GLP-1 signaling, enhanced satiety, improved insulin sensitivity, and increased fat oxidation. Unlike other pharmaceutical peptide based GLP-1 agonists, such as semaglutide or lariaglutide, (or double peptide agonists (GLP-1/GIP), such as tirzepatide, and even triple peptide agonists (GLP-1/GIP/CCG), such as retatrutide) which directly override receptors with high-potency stimulation, OEA, in accordance with the present invention, may work within physiological signaling pathways. It may act to encourage the body to regulate appetite through its own mechanisms rather than pharmacologically forcing the pathway. As such, the triple-agonist composition 10, may result in lower incidence of nausea, less gastric distress, no forced gastric emptying delay, and no receptor overstimulation.

Cellular Energy Reset via AMPK: Berberine-Metabolic Master Switch

Berberine activates AMPK, the master regulator of cellular energy balance. AMPK activation may, improve glucose uptake (GLUT4 translocation), reduce hepatic gluconeogenesis, inhibit fat synthesis, improves lipid profiles, enhance insulin sensitivity

Together, OEA (increases fatty acid oxidation and Berberine activates AMPK to increase fuel utilization) may create a coordinated shift toward fat burning and improved metabolic flexibility. Instead of suppressing appetite (like pharmaceutical GLP-1s), the body may also be: burning stored fat more efficiently, improving insulin signaling, clearing LDL more effectively, reduce hepatic fat accumulation. Appetite control in combination with metabolic acceleration may result in amplified impact.

Glucose Entry Control: Gymnema sylvestre: Reducing Incoming Glucose Load.

Use of GS in the triple-agonist composition 10 may provide the following, compete with glucose at intestinal receptors, reduce postprandial spikes, support insulin secretion, improve lipid markers, and provide antioxidant protection. In combination, the actions may include, Gymnema sylvestre for reducing how much glucose enters the bloodstream, berberine for improving how efficiently glucose is used and improving cellular energy balance, OEA for improving fat oxidation, insulin sensitivity and satiety. Use of the triple-agonist composition 10 may result in GLP-1 being naturally stimulated and inflammation markers being reduced.

Use of the triple-agonist composition 10 creates a closed metabolic circuit of improved glucose handling, reduced fat storage, and enhanced satiety signaling.

Use of the triple-agonist composition 10 may have several important effects: appetite reduction, caloric intake decreases naturally, blunted glucose spikes, insulin sensitivity improvements, increased fat oxidation, inflammation declines, and moderated dopamine-driven cravings.

Each pathway reinforces the others. Reduced glucose spikes→improved insulin sensitivity→enhanced fat oxidation→reduced liver fat→improved GLP-1 signaling→better appetite control.

This is systems-level metabolic modulation rather than single-receptor stimulation and offers advantages not associated with similar pharmaceutical peptide treatments. Pharmaceutical GLP-1 agonists work primarily by: 1) potent GLP-1 receptor agonism, 2) delaying gastric emptying, 3) strong appetite suppression. These pharmaceutical peptides are highly effective but commonly associated with various side effects, such as nausea, vomiting, constipation, loss of lean mass in some individuals, and gastrointestinal discomfort.

The triple-agonist composition 10 differs in several key ways. First, regarding physiological vs pharmacological signaling, OEA may stimulate endogenous GLP-1 production rather than artificially activating receptors at supra physiological levels. Second, regarding multi-pathway vs single-pathway, pharmaceutical GLP-1s focus heavily on appetite suppression. This combination targets appetite, insulin sensitivity, fat oxidation, glucose entry, lipid clearance, and inflammation simultaneously. Finally, regarding lower side effect burden, because the triple-agonist composition 10 mechanisms are modulatory rather than receptor-overriding, use of the triple-agonist composition 10 is expected to result in nausea being significantly less common, reduced gastric distress, forced gastric paralysis effect being absent, absence of abrupt appetite shutdown. Instead of shutting metabolism down, the triple-agonist composition 10 is believed to improve metabolic efficiency.

The present invention is further described by the following non-limiting examples. The following examples illustrate embodiments of routes of administration and forms of the composition. The forms and/or routes include, but are limited to tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration. The compositions may also be developed for inhalational, oral, rectal, vaginal, parenteral, topical, transdermal, pulmonary, intranasal, buccal, ophthalmic, intrathecal, intravenous or any route of administration.

The dosage form of the present invention may include either immediate or controlled release forms. Each composition can be formulated and manufactured by procedures known to one of skill in the art, and may include 100% composition or a mixture of one or more pharmaceutically acceptable excipients or pharmaceutically acceptable carriers.

In certain embodiments, the triple-agonist composition 10 may be formulated according to Table 1:

| Ingredient/Component | Concentration |
|---|---|
| a first compound that targets | an effective amount |
| Glucagon-like peptide 1 (GLP-1) | |
| receptors; | |
| a second compound that targets | an effective amount |
| Glucagon (GCG) receptors; and | |
| a third compound that targets | an effective amount |
| Glucose-dependent Insulinotropic | |
| Polypeptide (GIP) receptors. | |

In certain embodiments, the triple-agonist composition 10 may be formulated according to Table 2:

| Ingredient/Component | Concentration |
|---|---|
| a first compound that targets | an effective amount |
| Glucagon-like peptide 1 (GLP-1) | |
| receptors; | |
| a second compound that targets | an effective amount |
| Glucagon (GCG) receptors; and | |
| a third compound that targets | an effective amount |
| Glucose-dependent Insulinotropic | |
| Polypeptide (GIP) receptors. | |
| pharmaceutically acceptable | effective amounts |
| excipient | |

In certain embodiments, the triple-agonist composition 10 may be formulated according to Table 3:

| Ingredient/Component | Concentration |
|---|---|
| a first compound, OEA that | an effective amount |
| targets Glucagon-like peptide 1 | |
| (GLP-1) receptors; | |
| a second compound, Berberine | an effective amount |
| that targets Glucagon (GCG) | |
| receptors; and | |
| a third compound, GS, that | an effective amount |
| targets Glucose-dependent | |
| Insulinotropic Polypeptide (GIP) | |
| receptors. | |

In certain embodiments, the triple-agonist composition 10 may be formulated according to Table 4:

| Ingredient/Component | Concentration |
|---|---|
| a first compound, OEA, that | an effective amount |
| targets Glucagon-like peptide 1 | |
| (GLP-1) receptors; | |
| a second compound, Berberine, | an effective amount |
| that targets Glucagon (GCG) | |
| receptors; and | |
| a third compound, GS, that | an effective amount |
| targets Glucose-dependent | |
| Insulinotropic Polypeptide (GIP) | |
| receptors. | |
| pharmaceutically acceptable | effective amounts |
| excipient | |

In certain embodiments, the triple-agonist composition 10 may be formulated according to Table 5:

| Ingredient/Component | Concentration |
|---|---|
| OEA | about 100 mg-about 600 mg |
| Berberine | about 50 mg-about 1200 mg |
| GS | about 200 mg-about 900 mg |

In certain embodiments, the triple-agonist composition 10 may be formulated according to Table 6:

| Ingredient/Component | Concentration |
|---|---|
| OEA | about 100 mg-about 600 mg |
| Berberine | about 50 mg-about 1200 mg |
| GS | about 200 mg-about 900 mg |
| pharmaceutically acceptable | effective amounts |
| excipient | |

In certain embodiments, the triple-agonist composition 10 may be formulated according to Table 7:

| Ingredient/Component | Concentration |
|---|---|
| OEA | about 100 mg-about 600 mg |
| Berberine | about 50 mg-about 1200 mg |
| GS | about 200 mg-about 900 mg |
| pharmaceutically acceptable | effective amounts |
| excipients: | |
| Magnesium Stearate, | |
| Dicalcium Phosphate Dehydrate, | |
| Silicon Dioxide, | |
| Bamboo Leaf extract | |

In certain embodiments, the triple-agonist composition 10 may be formulated according to Table 8:

| Ingredient/Component | Concentration |
|---|---|
| OEA | about 100 mg-about 600 mg |
| Berberine | about 50 mg-about 1200 mg |
| GS | about 200 mg-about 900 mg |
| Magnesium Stearate; 5 | About 12 mg |
| Dicalcium Phosphate Dehydrate | About 190 mg |
| Silicon Dioxide; | About 7 mg |
| Bamboo Leaf extract | About 50 mg |

### Clinical Evaluation of Metabolic and Anthropometric Outcomes

The data demonstrate consistent improvements in weight, body mass index, and select metabolic and inflammatory markers without evidence of clinical deterioration or safety concerns.

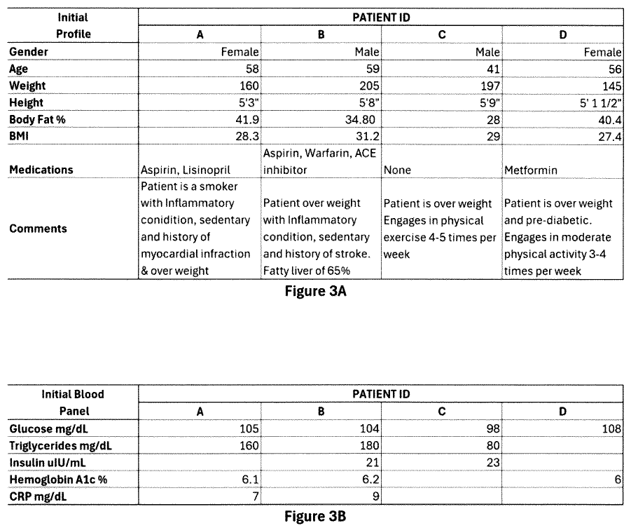

Study Overview: The triple-agonist composition 10, in accordance with embodiments of the invention described herein, was administered to 4 subjects, subject 1, Patient ID. A, a 58-year-old female, subject 2, Patient ID. B, a 59-year-old male, subject 3, Patient ID C, a 49-year-old male, and subject 4, Patient ID D, a 56-year-old female. Referring to FIGS. 3A and 3B, various physiological (FIG. 3A) and metabolic characteristics (FIG. 3B) for each subject, pre-administration of the triple-agonist composition, are provided. The triple-agonist composition 10 administered, in capsule form, comprised:
Oleoylethanolamide (OEA), 250 mg,
Berberine HCL, 500 mg
Gymnema sylvestre, 400 mg
Magnesium Stearate, 12 mg
Dicalcium Phosphate Dehydrate, 189.22 mg
Silicon Dioxide, 7 mg
Bamboo Leaf extract 50 mg
   Each patient, Patient ID. A, Patient ID. B, Patient ID C, and Patient ID D, took the triple-agonist composition 10, once a day, for eight weeks. Endpoints evaluated included, body weight, body mass index (BMI), body fat percentage, hemoglobin A1c, fasting insulin, lipid markers, C reactive protein, and subjective appetite and energy reports.

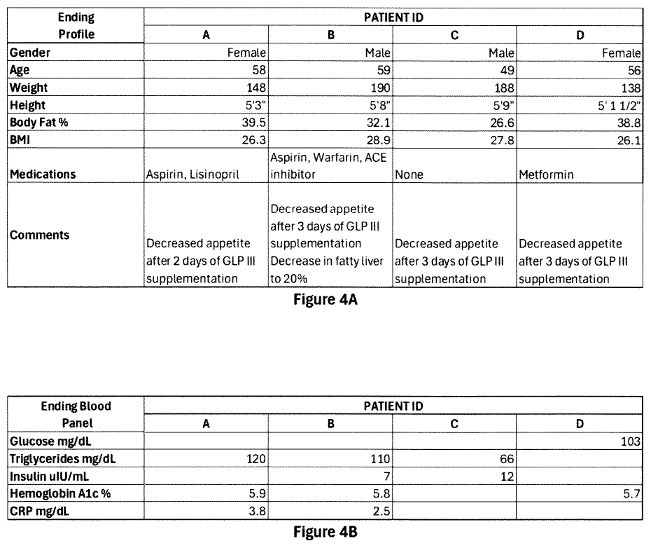

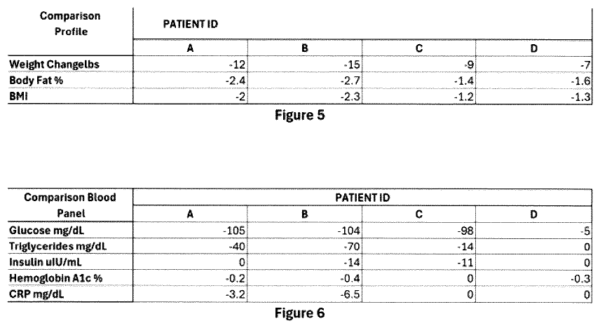

Various physiological characteristics (FIG. 4A) various metabolic characteristic (FIG. 4B) were tested after the eight-week time period. As shown FIG. 5, each of the subjects 1) lost weight (12 lbs., 15 lbs., 9 lbs., and 7 lbs., respectively), and 2) BMI in each subject was lowered. As shown in FIG. 6, taking the triple-agonist composition 10 for eight weeks resulted in changes in glucose (all reduced), triglycerides (all reduced), hemoglobin (reduced in three subjects) and CRP (reduced in two subjects) were lower than initial testing values.

Anthropometric Outcomes: All subjects experienced measurable weight reduction. The mean weight loss was 10.75 lbs. over eight weeks. Additional findings include: BMI decreased in all participants, recorded body fat percentages declined where data was available, and appetite suppression was reported within several days of initiation in all subjects. These changes suggest a consistent physiologic effect on satiety signaling and caloric intake.

Glycemic and Metabolic Findings: Metabolic markers demonstrated favorable trends. Hemoglobin A1c decreased in three of four subjects. Fasting insulin decreased in subjects with elevated baseline levels. No participant exhibited worsening glycemic control. These observations suggest a potential improvement in insulin sensitivity and metabolic efficiency.

Lipid Parameters: Baseline lipid abnormalities were present in multiple participants. Available follow up data indicated stability or improvement in lipid values. No subject demonstrated adverse lipid shifts, supporting a favorable metabolic safety profile during the observation period.

Inflammatory Markers: Participants with elevated baseline inflammatory markers demonstrated reductions in C reactive protein values. The magnitude of decline in at least one subject was substantial, suggesting possible systemic anti-inflammatory effects associated with the intervention.

Safety and Tolerability Assessment: No adverse clinical events were documented. Subjects consistently reported: increased energy, reduced cravings, and improved satiety. No medication discontinuations or intolerances were reported during the study period.

The directional consistency of improvement across independent physiologic domains including weight, metabolic regulation, and inflammatory status was notable. Concordant improvements across multiple objective endpoints in all participants reinforced the likelihood of a true biologic response. The magnitude of weight loss over eight weeks was clinically meaningful and aligns with physiologic responses seen in interventions that affect appetite regulation pathways.

All patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A triple-agonist composition targeting three different hormone receptors comprising:
an effective amount of a first compound that targets Glucagon-like peptide 1 (GLP-1) receptors;
an effective amount of a second compound that targets Glucagon (GCG) receptors; and
an effective amount of a third compound that targets Glucose-dependent Insulinotropic Polypeptide (GIP) receptors.

2. The triple-agonist according to claim 1, further including one or more pharmaceutically acceptable excipients.

3. The triple-agonist according to claim 1, wherein,
said first compound is Oleoylethanolamide, its pharmaceutically acceptable salts, of between about 100 mg and about 600 mg.

4. The triple-agonist according to claim 1, wherein said second compound is berberine, its pharmaceutically acceptable salts, of between about 50 mg to about 1200 mg.

5. The triple-agonist according to claim 1, wherein said third compound is Gymnema sylvestre, its pharmaceutically acceptable salts, of between about 200 mg to about 900 mg.

6. The triple-agonist according to claim 1, wherein said composition is delivered in the form of a pill, a capsule, an injectable, or a swallowable liquid.

7. A triple-agonist composition targeting three different hormone receptors comprising:
Oleoylethanolamide, its pharmaceutically acceptable salts, of between about 100 mg to about 600 mg;
Berberine, its pharmaceutically acceptable salts, of between about 50 mg to about 1200 mg;
Gymnema sylvestre, its pharmaceutically acceptable salts, of between about 200 mg to about 900 mg; and
wherein said composition is in a deliverable form to a subject in need.

8. The triple-agonist according to claim 7, further comprising one or more pharmaceutically acceptable excipients or one or more pharmaceutically acceptable carriers.

9. The triple-agonist according to claim 7, wherein said composition is delivered orally.

10. A method of controlling metabolic syndrome conditions or preventing, treating, or ameliorating metabolic diseases or disorders comprising:
delivering, to a subject in need thereof, an effective amount of a triple-agonist composition targeting three different hormone receptors,
said triple-agonist composition comprising:
an effective amount of a first compound that targets Glucagon-like peptide 1 (GLP-1) receptors, said first compound is Oleoylethanolamide, or its pharmaceutically acceptable salts;
an effective amount of a second compound that targets Glucagon (GCG) receptors, said second compound is berberine, or its pharmaceutically acceptable salts; and
an effective amount of a third compound that targets Glucose-dependent Insulinotropic Polypeptide (GIP) receptors, said third compound is Gymnema sylvestre, or its pharmaceutically acceptable salts.

11. The method according to claim 10, wherein,
Oleoylethanolamide is at a concentration of between about 100 mg to about 600 mg;
Berberine, or its pharmaceutically acceptable salts, is at a concentration of between about 50 mg to about 1200 mg; and
Gymnema sylvestre, or its pharmaceutically acceptable salts, is at a concentration of between about 200 mg to about 900 mg.

12. The method according to claim 10, wherein, triple-agonist composition is delivered 1-3 times daily.

13. The method according to claim 10, wherein, said triple-agonist composition is delivered to a subject suffering from obesity, a subject in need of weight loss, a subject in need of appetite reduction, or a subject in need of increase satiety.

14. The method according to claim 10, wherein, said triple-agonist composition is delivered to a subject who requires weaning from drugs that act on GLP or other G receptors

15. The method according to claim 10, wherein, said triple-agonist composition is delivered to said subject orally as a pill, a tablet, or a capsule.
